(19) United States
(12) Patent Application Publication
(10) Pub. No.: US 2005/0273118 A1
(43) Pub. Date: Dec. 8, 2005

(54) Epilator

(76) Inventors: Andreas Hillenmeier, Ingelheim (DE); Alexander SChoffmann, Graz (AT)

Correspondence Address: Robert J Schneider;Chapman and Cutler, 111 West Monroe Street, Chicago, IL, 60603, US

(21) Appl. No.: 10/520,227

(22) PCT Filed: Jul. 10, 2003
(86) PCT No.: PCT/AT03/00195

§ 371 Date: Jan. 4, 2005

(30) Foreign Application Priority Data
Jul. 11, 2002 (AT) GM 463/2002

(51) Int. Cl. A61B 17/50

(52) U.S. Cl. 606/133

(57) ABSTRACT

In an epilating device including a rotary cylinder (1) capable of being set in rotation about its axis (2), clamping means (7) for gripping and removing hairs are arranged about the circumference of the same in a manner offset in the circumferential direction, and the actuating means for the clamping means (7) are designed arranged in a manner that at least two clamping means (7) offset in the circumferential direction are each actuated at the same time.

The invention relates to an epilating device including a rotary cylinder capable of being set in rotation about its axis and having clamping means arranged about its circumference in a manner offset in the circumferential direction for gripping and removing hairs.

Such epilating devices or rotary cylinders for epilating devices are known from the prior art such as, for instance, WO 98/05234 or U.S. Pat. No. 5,196,021. In most cases, several clamping means are distributedly arranged about the circumference of the rotary cylinder, whereby the arrangement of the clamping means is usually realized in several rows parallel with the axis of rotation of the rotary cylinder such that all of the clamping means of a row will pass through a plucking zone at one and the same time. The clamping means arranged in a row are thereby actuated together simultaneously, thus forming a linear plucking zone on the user's skin.

In the epilating device according to U.S. Pat. No. 5,196,021, the rotary cylinder is comprised of a plurality of discs comprising radially outwardly projecting clamping elements, wherein two clamping elements of two neighbouring discs are each movable towards each other, thus forming a clamping means. In order to actuate the clamping means, the individual clamping elements comprise projections which extend in the axial direction of the rotary cylinder and are guided past end-side press rolls during the rotation of the rotary cylinder in a manner that two clamping elements are each moved towards each other by double actuation and brought into mutual clamping contact in a plucking zone of the device. The clamping elements of all discs constituting the rotary cylinder are relatively arranged in a manner that only one pair of clamping elements will each be in a plucking zone of the epilating device.

The clamping means in those known epilating devices are, thus, always actuated in a manner that a point-shaped and, at best, linear plucking zone extending in the direction of the axis of rotation of the rotary cylinder will result on the user's skin. The efficiency of epilation is, however, substantially reduced, because the safe and effective seizure of the body hairs to be plucked out will only be guaranteed if the epilating device is held by the user at a predefined angle relative to the skin surface and the clamping means are actuated exactly as the clamping means are getting into contact with the user's skin. It has, however, turned out in practice that the observance of a predefined constant orientation of the epilating device relative to the skin is almost impossible such that it cannot be avoided that the rotary cylinder is placed on the skin with a portion in which the clamping means are not yet closed, only incompletely closed or no longer closed and, therefore, ineffective. The present invention, therefore, aims to provide an epilating device which ensures efficient epilation even if the epilating device is held relative to the skin at varying angles following the natural course of movement during use.

To this end, the epilating device of the initially mentioned kind is essentially characterized according to the invention in that the actuating means for the clamping means are designed and arranged in a manner that at least two clamping means offset in the circumferential direction are each actuated at the same time. Due to the fact that, as opposed to the known epilating devices, the clamping means are not actuated along a linear plucking zone extending in the direction of the axis of the rotary cylinder, but actuation is effected in a manner that at least two clamping means offset in the circumferential direction are each actuated simultaneously, provides for an effective plucking zone that extends over an enlarged region, viewed in the circumferential direction of the rotary cylinder. In doing so, at least one of the at least two clamping means will effectively enter into action as a function of the respective epilating device angle relative to the skin selected by the user. Since the at least two clamping means which are actuated simultaneously are not located on a common axially parallel line or generatrix of the rotary cylinder, the relative distance of the simultaneously actuated clamping means is accordingly enlarged, thus causing a reduction of unpleasant sensations to be observed subjectively.

According to a preferred configuration, the offset of the simultaneously actuated clamping means is between 3° and 45°, preferably 320. It goes without saying that also a larger offset in the circumferential direction is conceivable. The preferred angle indicated produces a plucking zone that extends at a central angle of about 500 about the circumference of the rotary cylinder so as to facilitate the plucking out of hairs. The extent of the offset in this case may be chosen such that an overlap of the simultaneously actuated clamping means will result in the axial direction of the rotary cylinder, or such that the simultaneously actuated clamping means will immediately follow one another in the circumferential direction.

The epilating device according to the invention, thus, requires the respective at least two clamping means offset in the circumferential direction and passing through the plucking zones to be actuated simultaneously during the rotation of the rotary cylinder. To this end, it is advantageous that the individual clamping means are actuatable individually and separately from one another, which will naturally result in elevated structural expenses if a plurality of clamping means is to be arranged along the circumference of the rotary cylinder. In order to obtain a simpler construction nevertheless, the configuration preferably is further developed in a manner that the respective at least two simultaneously actuated clamping means offset in the circumferential direction are arranged to be offset in the axial direction. In this embodiment, the simultaneously actuated clamping means are, thus, arranged along a line extending in a manner inclined relative to the axis of the rotary cylinder. It has, thus, become feasible to allocate to the jointly actuated clamping means common structural components intended, for instance, for the guidance, mounting, actuation and return of the clamping means, and in this context the configuration may, for instance, be devised such that the movable clamping elements of the respective clamping means actuated simultaneously are associated with a common spring element, against the force of which the clamping elements are each displaceable.

Further simplification results from that fact that, as in correspondence with a preferred further development, the clamping means are each comprised of a clamping element fixed relative to the rotary cylinder and a movable clamping element capable of being pressed against the fixed clamping element. In this case, just one clamping element need be actuated, and so the actuating mechanism can be designed in a substantially simpler manner. The fixed clamping elements in this case may each be formed by a side wall of a hole provided in the rotary cylinder, into which one movable clamping element each immerses. This ensures the optimum integration of the clamping means in the rotary cylinder and hence a particularly skin-protecting epilation, it merely having to be taken care that the movable clamping elements do not project out of the holes of the rotary cylinder beyond the external jacket of the rotary cylinder. Skin irritations due to sharp-edged clamping elements are thereby avoided.

In order to enable the separate actuation of the individual clamping means, the actuating means in a preferred manner comprise coupling members extending in the axial direction of the rotary cylinder and cooperating with the clamping means. The coupling members extending in the axial direction of the rotary cylinder in this case serve to transmit the actuation stroke from the end side of the rotary cylinder to the individual clamping means, wherein each clamping means is associated with a separate coupling member so as to ensure the separate actuation of all clamping means. The configuration in this case preferably is further developed in that the coupling members are designed as slides movably guided in the axial direction of the rotary cylinder, wherein the movable clamping elements are each coupled with one slide in an angularly firm manner. In this configuration, the actuation stroke is directly transmitted to the clamping elements on account of the angularly firm connection of the clamping elements with the coupling member such that a particularly advantageous and direct force transmission results. The movable clamping elements in this context are always guided in parallel with the fixed clamping element, thus obtaining a linear contact between the two clamping elements of one clamping means. This ensures the particularly firm gripping and reliable removal of the hairs to be plucked out, while avoiding pivotal movements and clamping or jamming of the individual clamping elements.

The slides can each be slidingly mounted on two mounting rods extending in the axial direction, actuating elements neighbouring in the circumferential direction comprising one common mounting rod at most. By using continuous mounting rods, the risk of jamming due to eccentric stress is avoided. Furthermore, this enables several slides to be guided on the same two continuous mounting rods, thus rendering feasible a further reduction of the number of structural components. In doing so, the slides of the respective clamping means actuated simultaneously can be guided on two common mounting rods, wherein a spring element acting in the axial direction is arranged between these slides and wherein, furthermore, at least one of these slides includes a region offset in the direction of rotation of the rotary cylinder and at least another one of these slides includes a region offset against the direction of rotation of the rotary cylinder, with which offset regions the movable clamping element is each coupled or connected. This construction enables the two simultaneously actuated clamping means to be mounted on a common axial guide, yet be arranged in a manner offset in the circumferential direction.

Suitable control elements must be provided to actuate and control the individual clamping means, and the configuration advantageously is designed such that the actuating means comprise control elements arranged on the end sides of the rotary cylinder and cooperating with the coupling members to actuate the clamping means. Preferably, the configuration in this respect is devised such that on each end side of the rotary cylinder a press roll is arranged, onto which the coupling elements run, whereby one of the press rolls is arranged to be offset relative to the opposite press roll in the circumferential direction of the rotary cylinder, said offset arrangement of the actuation-stroke-initiating press rolls enabling clamping means that are arranged to be offset in the circumferential direction to be actuated simultaneously. The same result can naturally be obtained also the other way round in that, for instance, the contact surfaces of the coupling members, which cooperate with the press rolls, are arranged to be offset in the circumferential direction of the rotary cylinder, in which latter case an offset arrangement of the press rolls themselves is not required. Departing from the first-mentioned alternative, in which the press rolls are arranged to be offset in the circumferential direction, the extent of the offset of the press rolls determines the effective plucking zone of the epilating device, and in this context the configuration advantageously is devised such that the offset of the press rolls is <600, preferably 32°.

In the following, the invention will be explained in more detail by way of an exemplary embodiment schematically illustrated in the drawing. Therein,

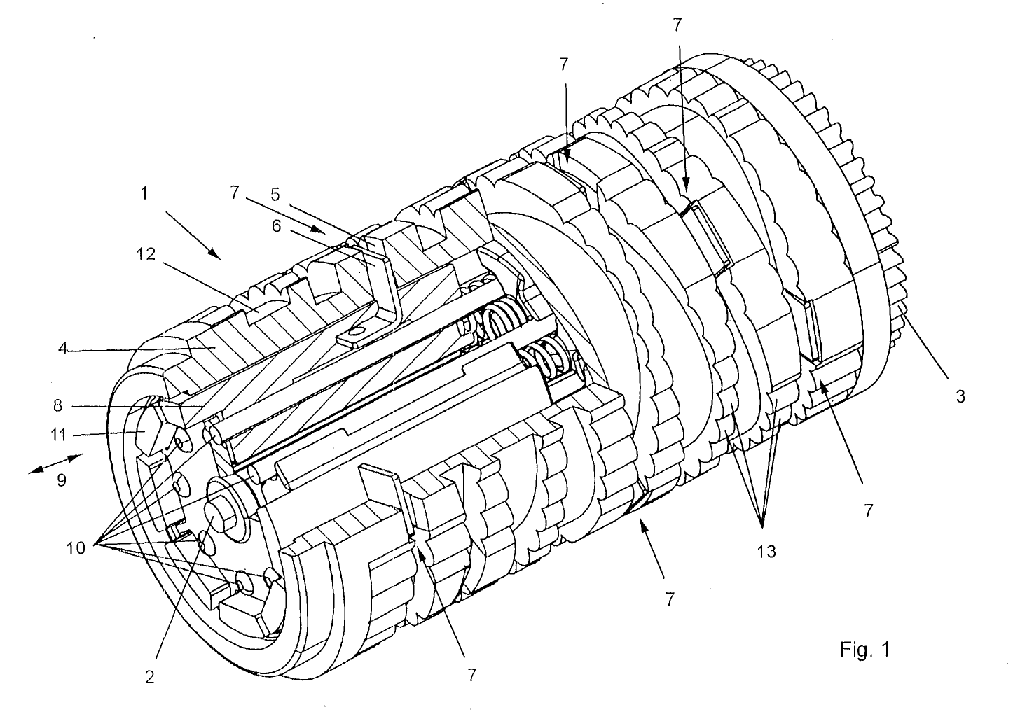

FIG. 1 is a perspective view of the rotary cylinder of an epilating device;

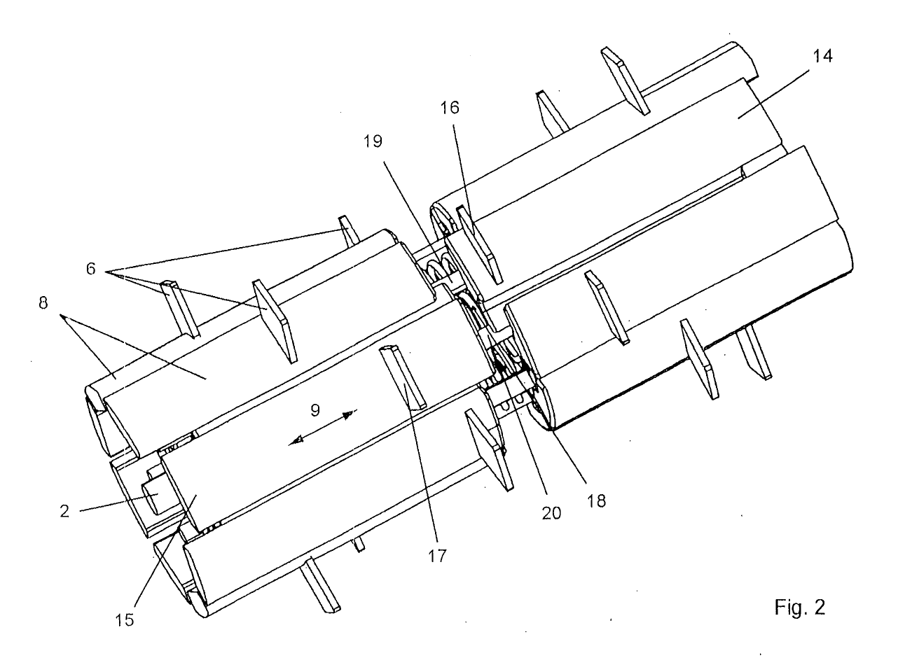

FIG. 2 is a perspective view of the plucking cylinder without cylinder jacket;

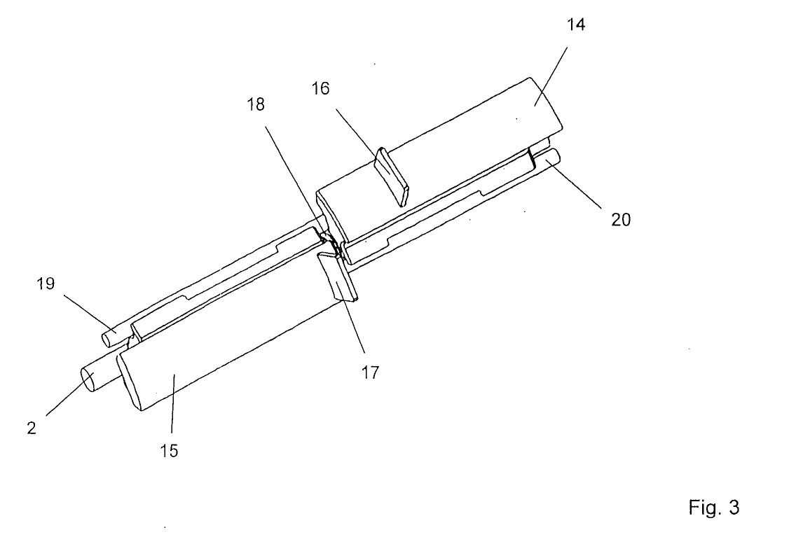

FIG. 3 is a partial view of FIG. 2; and

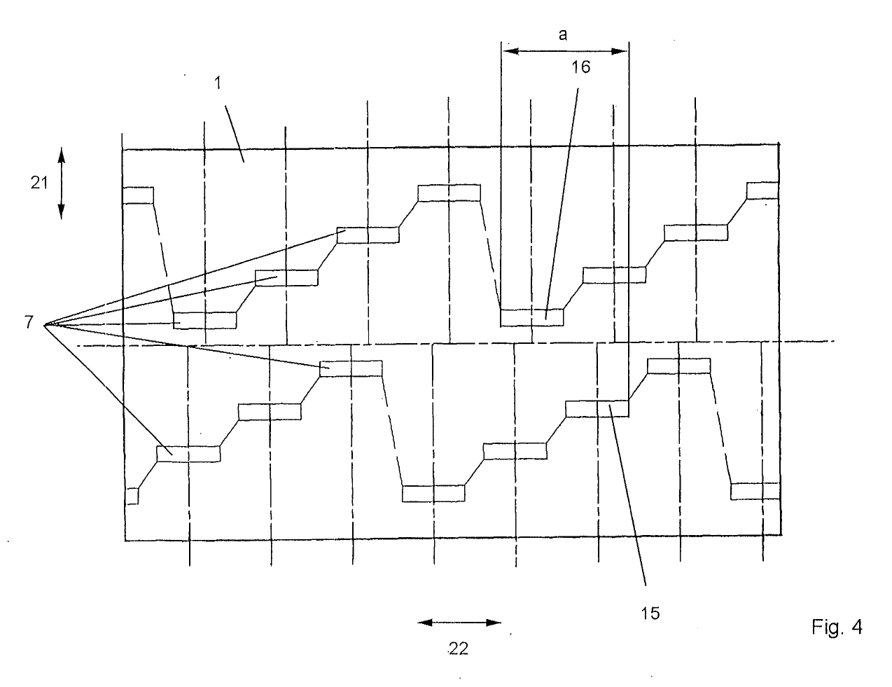

FIG. 4 is a schematic illustration of the developed view of a rotary cylinder.

From FIG. 1, a rotary cylinder 1 is apparent, which is mounted so as to be rotatable about an axis 2. The actuation of the cylinder 1 is realized by the aid of a pinion 3 arranged on one of the end walls of the cylinder 1 and cooperating with an actuating unit not illustrated in detail. In the jacket 4 of the cylinder are provided holes 5 in which clamping elements formed by angle sheets 6 immerse. The clamping elements 6 are movably arranged, each forming clamping means 7 together with the respective side walls of the holes 5. The movable clamping elements 6 are rigidly connected with slides 8 extending in the direction of the axis 2. Each slide 8 is connected with a single clamping sheet 6 such that each clamping means can be actuated separately by displacing the slide 8 along double arrow 9. Each slide 8 is guided on the respective two of mounting rods 10 uniformly distributed about the circumference. The actuation of the slides 8 is effected via a press roll which is arranged on each end side of the cylinder 1 and on which the slides 8 run with their end faces 11 so as to cause the displacement of the slides 8.

The jacket 4 of the cylinder 1, furthermore, is provided with socalled thread-in grooves 12, one thread-in groove being allocated to each clamping means 7. The thread-in grooves 12 during the rotation of the cylinder 1 serve to direct the hairs to be plucked out into paths that enable their feeding to the clamping means 7. To this end, the thread-in grooves 12 open within the holes 5, said thread-in grooves 12 being designed in a manner constantly narrowing towards the clamping means 7. Moreover, the jacket 4 of the cylinder 1 about its entire circumference is formed with ribs 13 that enter into contact with the skin surface during the rotation of the cylinder 1, thus causing a stimulation of the skin in a manner that pain irritations induced by the plucking out of hairs will be overlaid and less markedly noticed subjectively. The result is a substantial pain alleviation during hair removal.

FIG. 2 depicts the rotary cylinder 1 without its outer jacket 4, the arrangement of the slides 8 with the clamping elements 6 thus being more clearly visible. The actuation of the individual clamping elements is now effected in that two clamping means arranged to be offset in the circumferential direction are each actuated at the same time.

In FIG. 2, as well as in the detailed illustration according to FIG. 3, the slides 14 and 15 together with their pertinent clamping elements 16 and 17 are illustrated as actuated simultaneously and hence in a mutually displaced position. As already mentioned, the displacement of the slides 14 and 15 is effected via end-side press rolls not illustrated in detail, and against the force of a spring 18 arranged between the slides 14 and 15. After a rotation of the cylinder 1 about an angle corresponding to the width of a slide, the respectively neighbouring slides are actuated by the press rolls with the return of the previously displaced slides 14 and 15 being ensured by the force of the spring 18. The slides 14 and 15 are guided on two common mounting rods 19 and 20, as is illustrated even more clearly in FIG. 3. The slides 14 and 15 each comprise a base region by which they are guided along the mounting rods 19 and 20, and an upper region on which the clamping elements 16 and 17 are arranged and which is offset in the circumferential direction.

FIG. 4 schematically indicates a developed view of the cylinder 1 with the arrangement of the individual clamping means 7 being even more clearly visible. The axial direction of the rotary cylinder 1 in the instant case is indicated by double arrow 21 and its circumferential direction by double arrow 22. Again, the clamping elements 15 and 16 which are arranged to be offset in the circumferential direction 22 and actuated simultaneously are illustrated as examples. During the rotation of the rotary cylinder 1, the clamping elements consecutively arranged in the circumferential direction 22 are actuated one after the other with two clamping means arranged to be offset in the circumferential each being actuated at the same time. This type of actuation provides a plucking zone that corresponds to distance a and hence is substantially larger than in epilating devices in which the clamping means are actuated along a line or row extending in the axial direction 21.

1. An epilating device comprising a rotary cylinder capable of being set in rotation about its axis and having clamping means arranged about its circumference in a manner offset in the circumferential direction for gripping and removing hairs, wherein actuating means for the clamping means (7) are designed and arranged in a manner that at least two clamping means (7) offset in the circumferential direction are each actuated at the same time.

2. An epilating device according to claim 1, wherein the offset of the simultaneously actuated clamping means (7) is between 3° and 45°.

3. An epilating device according to claim 1, wherein the respective at least two simultaneously actuated clamping means (7) offset in the circumferential direction are arranged to be offset in the axial direction.

4. An epilating device according to claim 1, wherein the clamping means (7) are each comprised of a clamping element (5) fixed relative to the rotary cylinder (1) and a movable clamping element (6) capable of being pressed against the fixed clamping element (5).

5. An epilating device according to claim 4, wherein the fixed clamping elements (5) are each formed by a side wall of a hole provided in the rotary cylinder (1), into which one movable clamping element (6) each immerses.

6. An epilating device according to claim 1, wherein the actuating means comprise coupling members extending in the axial direction (2) of the rotary cylinder (1) and cooperating with the clamping means (7) to actuate the same.

7. An epilating device according to claim 6, wherein the coupling members are designed as slides (8) movably guided in the axial direction (2) of the rotary cylinder (1), and wherein movable clamping elements (6) are each coupled with one slide (8) in an angularly firm manner.

8. An epilating device according to claim 7, wherein the slides (8) are each slidingly mounted on two mounting rods (10) extending in the axial direction (2), slides (8) neighbouring in the circumferential direction comprising one common mounting rod (10) at most.

9. An epilating device according to claim 4, wherein the movable clamping elements (6) of the respective clamping means (7) actuated simultaneously are associated with a common spring element (18), against the force of which the clamping elements (6) are each displaceable.

10. An epilating device according to claim 7, wherein the slides (8) of the respective clamping means (7) actuated simultaneously are guided on two common mounting rods (10) with a spring element (18) acting in the axial direction being arranged between these slides (8), and that at least one of these slides (8) includes a region offset in the direction of rotation of the rotary cylinder (1) and at least another one of these slides (8) includes a region offset against the direction of rotation of the rotary cylinder (1), with which offset regions the movable clamping element (6) is each coupled or connected.

11. An epilating device according to claim 6, wherein the actuating means comprise control elements arranged on the end sides of the rotary cylinder (1) and cooperating with the coupling members to actuate the clamping means (7).

12. An epilating device according to claim 6, wherein on each end side of the rotary cylinder (1) a press roll is arranged, onto which the coupling members run, wherein one of the press rolls is arranged to be offset relative to the opposite press roll in the circumferential direction of the rotary cylinder (1).

13. An epilating device according to claim 12, wherein the offset of the press rolls is <60°.

14. An epilating device according to claim 1, wherein the offset of the simultaneously actuated clamping means (7) is about 32°.

15. An epilating device according to claim 3, wherein the clamping means (7) are each comprised of a clamping element (5) fixed relative to the rotary cylinder (1) and a movable clamping element (6) capable of being pressed against the fixed clamping element (5).

16. An epilating device according to claim 3, wherein the actuating means comprise coupling members extending in the axial direction (2) of the rotary cylinder (1) and cooperating with the clamping means (7) to actuate the same.

17. An epilating device according to claim 4, wherein the actuating means comprise coupling members extending in the axial direction (2) of the rotary cylinder (1) and cooperating with the clamping means (7) to actuate the same.

18. An epilating device according to claim 5, wherein the movable clamping elements (6) of the respective clamping means (7) actuated simultaneously are associated with a common spring element (18), against the force of which the clamping elements (6) are each displaceable.

19. An epilating device according to claim 6, wherein the movable clamping elements (6) of the respective clamping means (7) actuated simultaneously are associated with a common spring element (18), against the force of which the clamping elements (6) are each displaceable.

20. An epilating device according to claim 12, wherein the offset of the press rolls is about 32°.
